(19) United States
(12) Patent Application Publication
(10) Pub. No.: US 2026/0355288 A1
(43) Pub. Date: Nov. 12, 2026

(54) COMPOSITIONS COMPRISING INSULIN AGONISTS, AND SAID COMPOSITIONS FOR USE IN THE TREATMENT OF DIABETES OR OBESITY

(71) Applicant: Biotts Inc., Bielany Wroclawskie (PL)

(72) Inventors: Lukasz POGORZELEC, Wroclaw (PL); Pawel BIERNAT, Wroclaw (PL); Mieszko DYLINSKI, Wroclaw (PL); Dawid BURSY, Wroclaw (PL); Katarzyna PAULUS, Wroclaw (PL); Ewa MICEWICZ, Wroclaw (PL); Magdalena SZEWCZYK, Wroclaw (PL)

(73) Assignee: Biotts Inc., Bielany Wroclawskie (PL)

(21) Appl. No.: 19/514,950

(22) PCT Filed: Sep. 27, 2024
(86) PCT No.: PCT/IB2024/059477

§ 371 Date: Feb. 27, 2026

Related U.S. Application Data
(60) Provisional application No. 63/586,110, filed on Sep. 28, 2023.

(51) Int. Cl. A61K 38/28; A61K 9/00; A61K 9/70; A61K 47/10; A61K 47/14; A61K 47/18; A61K 47/28; A61K 47/44

(52) U.S. Cl. CPC A61K 38/28; A61K 9/0014; A61K 9/7023; A61K 47/10; A61K 47/14; A61K 47/186; A61K 47/28; A61K 47/44

(57) ABSTRACT

The present disclosure is directed to semi-solid compositions comprising insulin agonists, and to said compositions for use in the treatment of medical conditions, such as diabetes and obesity.

### FIELD OF THE INVENTION

The present disclosure is directed to semi-solid compositions comprising insulin agonists and methods of their use in treating medical conditions, such as diabetes and obesity.

### BACKGROUND OF THE INVENTION

Diabetes is a group of metabolic disorders characterized by elevated blood glucose levels, known as hyperglycemia. The increase in glucose results from defects in the production or action of insulin, which is secreted by beta cells in the pancreas. Chronic hyperglycemia leads to damage and dysfunction of various organs, such as the eyes, kidneys, nerves, heart, and blood vessels. There are different types of diabetes, including type 1, type 2, gestational diabetes, and others, which occur less frequently.

The most common type of diabetes is type 2 diabetes, which initially involves reduced tissue sensitivity to insulin, known as insulin resistance. This condition requires excessive insulin production, which eventually exceeds the pancreas' secretory capacity. In type 1 diabetes, beta cells in the pancreas are damaged, resulting in impaired or no insulin secretion.

In type 1 diabetes, an autoimmune process destroys beta cells that produce insulin, leading to insulin deficiency in the blood, while tissues remain sensitive to this hormone. Gestational diabetes is a result of hormonal changes associated with pregnancy. Both type 1 and type 2 diabetes have a genetic basis, with mutations in multiple genes responsible for their development, unlike monogenic diseases like MODY.

The principle of diabetes therapy involves treating all associated disorders. Achieving normal body weight, proper diet, treatment of common lipid disorders, arterial hypertension, and other cardiovascular diseases, as well as maintaining blood glucose levels as close as possible to non-diabetic values (normoglycemia), reduces the risk of diabetes-related complications.

Insulin therapy is a cornerstone of treatment for individuals with diabetes, particularly those with type 1 diabetes and some with type 2 diabetes. The goal of insulin therapy is to regulate blood glucose levels, maintaining them within a target range to prevent complications associated with high or low blood sugar levels. However, traditional methods for delivery of insulin, such subcutaneous administration, have their limitations and may not always be suitable for all patients. Moreover, many diabetes patients, particularly in the type 2 diabetes segment, are subject to so-called “needle-phobia,” i.e., a substantial fear of injecting themselves.

The transdermal delivery system has been widely investigated for the administration of different types of therapeutic agents. The transdermal route has many attractive properties for the administration of therapeutic agents, such as the relative ease of administration and the avoidance of the common problems associated with the oral route, such as first-pass metabolism. Although this method of administration has proven to be successful with various drugs, such as pain management agents, the delivery of proteins and peptides through the transdermal route is more challenging. This is mainly because proteins and peptides do not penetrate into the stratum corneum layer of the skin at a rate that allows for therapeutic application. Therefore, there exists a need in the art for improved delivery systems for insulin.

### BRIEF DESCRIPTION OF THE DRAWINGS

FIG. 1 shows a comparison of human insulin plasma concentrations in experimental rats after transdermal administration and subcutaneous injection.

### SUMMARY OF THE INVENTION

The present disclosure is directed to a pharmaceutical composition comprising an Insulin and a pharmaceutical carrier selected from dimethyl sulfoxide (DMSO), urea, propylene glycol, isopropanol, Vaseline, Adeps Suilus, oleic acid, Choline or their salts, L-Proline, Phosphatidylcholine, Tween 80, cetylic alcohol, cholesterol, vegetable oil, mineral oil, essential oils, or combinations thereof. In some aspects, the insulin is selected from the group consisting of NPH insulin, glargine insulin, degludec insulin, detemir insulin. In some aspects, the insulin is NPH insulin.

In some aspects, the composition described herein is formulated for transdermal delivery. In some aspects, the composition comprises a transdermal liquid formulation, a transdermal semisolid formulation, a transdermal polymer matrix formulation, or combinations thereof. In another aspect, the composition is in the form of a transdermal patch. In another aspect, the transdermal patch is selected from the group consisting of a reservoir patch, a micro reservoir patch, a matrix patch, a pressure sensitive adhesive patch, and an extended-release transdermal film.

The present disclosure is also directed to a method of transporting an Insulin through the skin and into systemic circulation comprising: administering to a subject in need of the treatment a formulation comprising a pharmaceutical composition described herein. In one aspect, the administration of the formulation comprising an Insulin and the pharmaceutically acceptable carrier comprises continuous administration of the formulation to the subject for one day, two days, three days, four days, five days, six days, seven days, eight days, nine days, ten days, eleven days, twelve days, thirteen days, or fourteen days.

The present disclosure is also directed to a method of treating diabetes in a subject, comprising transdermally administering a therapeutically effective amount of a pharmaceutical composition described herein to a subject in need thereof.

The present disclosure is also directed to a method of treating obesity in a subject, comprising transdermally administering a therapeutically effective amount of a pharmaceutical composition described herein to the subject in need thereof.

The present disclosure is also directed to the use of a pharmaceutical composition comprising an Insulin and a pharmaceutical carrier selected from dimethyl sulfoxide (DMSO), urea, propylene glycol, isopropanol, Vaseline, Adeps Suilus, oleic acid, Choline or their salts, Phosphatidylcholine, Tween 80, cetylic alcohol, cholesterol, vegetable oil, mineral oil or combinations thereof, in the treatment of diabetes.

### DETAILED DESCRIPTION OF THE INVENTION

The current disclosure is directed to a pharmaceutical formulation designed primarily for administration to mammals through transdermal or parenteral routes. This formulation encompasses a non-aqueous emulsion or dispersion, which, in addition to the hydrophilic phase, consists of the following components:

Active Ingredient: At least one therapeutically active protein or polypeptide.

Pharmaceutically Acceptable Emulsifier: At least one compound that serves as an emulsifying agent.

Oil Phase: in some aspects, this phase contains or mainly consists of esters derived from aliphatic hydroxy compounds with 1-12 carbon atoms and 1-4 alcoholic hydroxy groups, and aliphatic carboxylic acids with 8-24 carbon atoms and 1-3 carboxylic acid groups.

It should be noted that the therapeutic activity of the active ingredient within this formulation is such that the presence or the therapeutic effect of this ingredient becomes noticeable in the bloodstream in less than two hours, in some aspects for less than 1.5 hours and, in further aspects, within less than one hour after the commencement of transdermal administration.

Although, as previously mentioned, the present disclosure encompasses pharmaceutical formulations containing insulin without the need for emulsifiers or oil phases, there are notable advantages associated with the use of emulsifiers and/or oil phases. Without these components, the transdermal formulations could potentially lead to some skin irritation. However, the inclusion of emulsifiers and/or oil phases in the formulations appears to alleviate or prevent this issue.

Furthermore, the pharmaceutical formulations designed for transdermal administration under this invention can naturally incorporate a skin-irritation mitigation agent, if it is deemed advisable or required.

The active ingredient of choice is typically insulin. In this scenario, the pharmaceutical formulation described in this invention can be applied in the treatment of diabetes mellitus. The treatment involves administering the formulation, which contains an appropriate quantity of insulin for effectively managing diabetes mellitus. It is important to note that this formulation is inherently suitable for transdermal administration.

Another aspect of this invention involves a method for regulating blood glucose levels in diabetic patients. This method entails administering the pharmaceutical formulation described in this invention, specifically when the active ingredient is insulin. In a specific embodiment, this pharmaceutical formulation is administered to the patient through transdermal means, for example, by applying a matrix containing the pharmaceutical formulation onto the patient's skin. This matrix consists of a porous, absorbent, perforated, and flexible monolayer or multilayer solid support that has absorbed the pharmaceutical formulation described in this invention.

A significant advantage of the disclosed method is that when insulin is administered transdermally using this approach, it can be withdrawn at the discretion of the medical practitioner, for instance, if the patient experiences a hypoglycemic episode. This capability allows for the avoidance or mitigation of hypoglycemia or cyclical fluctuations between hypoglycemia and hyperglycemia, which are unfortunate but common outcomes when insulin is administered through conventional routes.

The disclosure provides insulin, for example Neutral protamine Hagedorn (NPH) insulin or Insulin glargine, insulin detemir, insulin degludec, containing formulations for transdermal administration. In general, it is an object of this disclosure to provide additional improved methods for reducing in subjects, including humans, in need of such treatment at least one of hyperglycemia, glycated hemoglobin A1c (HbA1c), glucose intolerance, insulin resistance. It is also an object of this disclosure to provide additional improved methods for reducing at least one condition, including type 1 and 2 diabetes.

Therefore, the disclosure is directed to a method for treating a mammalian subject with a condition that is also selected from the group consisting of insulin resistance syndrome and diabetes (both primary essential diabetes such as Type I Diabetes or Type II Diabetes and secondary nonessential diabetes), comprising administering to the subject an amount of a insulin compositions as described herein effective to treat the condition. In accordance with the methods described herein, the compositions are used to treat a symptom of diabetes or the chance of developing a symptom of diabetes, such as atherosclerosis, obesity, hypertension, hyperlipidemia, fatty liver disease, nephropathy, neuropathy, retinopathy, foot ulceration and cataracts, each such symptom being associated with diabetes, can be reduced. This disclosure also provides a method for treating hyperlipidemia comprising administering to the subject an amount of a biologically active agent as described herein effective to treat the condition. The compounds of the disclosure also provide a method for treating obesity comprising administering to the subject an amount of a pharmaceutical composition as described herein effective to treat the condition. This disclosure also provides a method for treating a condition selected from atherosclerosis or arteriosclerosis comprising administering to the subject an amount of a composition as described herein effective to treat the condition. The compositions of the disclosure are effective to treat hyperlipidemia, fatty liver disease, cachexia, obesity, atherosclerosis or arteriosclerosis whether or not the subject has diabetes or insulin resistance syndrome.

As used herein, “a,” an” and “the” include singular and plural referents unless the context clearly dictates otherwise. Thus, for example, reference to “an active agent” or “a pharmacologically active agent” includes a single active agent as well as two or more different active agents in combination, reference to “a carrier” includes mixtures of two or more carriers as well as a single carrier, and the like.

“Diabetes Mellitus” in the context of the present invention encompasses Type 1 Diabetes, both autoimmune and idiopathic and Type 2 Diabetes (together, “Diabetes”). The World Health Organization defines the diagnostic value of fasting plasma glucose concentration to 7.0 mmol/l (126 mg/dl) and above for Diabetes Mellitus (whole blood 6.1 mmol/l or 110 mg/dl), or 2-hour glucose level≥11.1 mmol/L (≥200 mg/dL). Other values suggestive of or indicating high risk for Diabetes Mellitus include elevated arterial pressure≥140/90 mm Hg; elevated plasma triglycerides (≥1.7 mmol/L; 150 mg/dL) and/or low HDL-cholesterol (<0.9 mmol/L, 35 mg/dl for men; <1.0 mmol/L, 39 mg/dl women); central obesity (males: waist to hip ratio>0.90; females: waist to hip ratio>0.85) and/or body mass index exceeding 30 kg/m²; microalbuminuria, where the urinary albumin excretion rate≥20 μg/min or albumin:creatinine ratio≥30 mg/g).

A “subject” or “patient” in the context of the present invention is preferably a mammal. The mammal can be a human, non-human primate, mouse, rat, dog, cat, horse, or cow, but are not limited to these examples. Mammals other than humans can be advantageously used as subjects that represent animal models of type 2 Diabetes Mellitus. A subject can be male or female. A subject can be one who has been previously diagnosed with or identified as suffering from or having type 1 or 2 Diabetes, one or more complications related to type 1 or 2 Diabetes, or a pre-diabetic condition, and optionally, but need not have already undergone treatment for the type 1 or 2 Diabetes, the one or more complications related to type 1 or 2 Diabetes. A subject can also be one who has been diagnosed with or identified as suffering from type 1 or 2 Diabetes, one or more complications related to type 1 or 2 Diabetes, but who show improvements in known Diabetes risk factors as a result of receiving one or more treatments for type 1 or 2 Diabetes, one or more complications related to type 1 or 2 Diabetes. Alternatively, a subject can also be one who has not been previously diagnosed as having Diabetes, one or more complications related to type 1 or 2 Diabetes. For example, a subject can be one who exhibits one or more risk factors for Diabetes, complications related to Diabetes, or a subject who does not exhibit Diabetes risk factors, or a subject who is asymptomatic for Diabetes, one or more Diabetes-related complications, or a pre-diabetic condition. A subject can also be one who is suffering from or at risk of developing Diabetes or obesity or a pre-diabetic condition. A subject can also be one who has been diagnosed with or identified as having one or more complications related to type 1 or 2 Diabetes condition as defined herein, or alternatively, a subject can be one who has not been previously diagnosed with or identified as having one or more complications related to type 1 or 2 Diabetes.

Patients with type 1 diabetes have an absolute deficiency of insulin, and thus, they require insulin therapy for survival. Many individuals with type 1 diabetes use MDI (Multiple Daily Injections), which involves multiple daily injections of insulin. These injections are typically done before meals to cover the rise in blood sugar that occurs after eating.

Basal-Bolus Regimen: The MDI approach often includes a combination of long-acting (basal) and short-acting (bolus) insulins. Long-acting insulin provides a steady background level of insulin throughout the day, while short-acting insulin is taken before meals to manage post-meal spikes in blood sugar. Some people with type 1 diabetes use insulin pumps, which deliver a continuous supply of insulin throughout the day, mimicking the basal insulin. They can also deliver bolus doses before meals.

In type 2 diabetes, insulin therapy is considered when other treatments, such as oral medications and lifestyle changes, do not adequately control blood sugar levels. Type 2 diabetes is a progressive condition, and over time, many individuals with this form of diabetes may require insulin as beta cell function deteriorates. When insulin is initiated in type 2 diabetes, it is often started as a once-daily injection of long-acting insulin. Over time, the regimen may be adjusted to include additional injections and different types of insulin. Some individuals with type 2 diabetes continue to take oral medications in addition to insulin to optimize blood sugar control.

Insulin therapy is a critical component of diabetes management, especially for those with type 1 diabetes and some with type 2 diabetes. It requires careful monitoring, adjustment, and a patient-centered approach to achieve and maintain good blood sugar control while minimizing the risk of complications.

Long-acting insulins, also known as basal insulins, are a type of insulin used in the management of diabetes. These insulins are designed to provide a slow and steady release of insulin into the bloodstream over an extended period. They mimic the background or baseline insulin production that occurs in the absence of meals and help keep blood sugar levels stable between meals and overnight.

Long-acting insulins primarily serve as basal or background insulin. Basal insulin covers the body's insulin needs when it is not actively digesting food. It helps prevent the liver from releasing too much glucose into the bloodstream and keeps fasting blood sugar levels in check.

Long-acting insulins have a slow onset of action, typically taking effect within 1 to 2 hours after injection. What sets them apart is their extended duration of action, which can last up to 24 hours or even longer, depending on the specific type of long-acting insulin. Unlike short-acting or rapid-acting insulins, long-acting insulins are designed to have minimal to no pronounced peak. This means that they provide a relatively constant level of insulin in the bloodstream throughout the day and night.

Long-acting insulins are often administered once daily, although some formulations may require twice-daily dosing for individuals who have higher insulin needs. The choice of dosing frequency depends on the type of long-acting insulin and the individual's specific needs. A range of different approaches have been used for modifying the structure of insulin compounds in order to provide a different duration of action in vivo. Insulin useful for the present invention include, but are not limited to those listed below.

In one aspect, the insulin is selected from the group consisting of NPH insulin, degludec insulin, galergine insulin. In one aspect, insulin is NPH insulin. In one aspect, the present invention encompasses pharmaceutically acceptable salts of insulin. Such salts include pharmaceutically acceptable pharmaceutically acceptable metal salts.

The term “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g., γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), β-alanine, 3-aminomethyl benzoic acid, anthranilic acid.

The 22 proteogenic amino acids are: Alanine, Arginine, Asparagine, Aspartic acid, Cysteine, Cystine, Glutamine, Glutamic acid, Glycine, Histidine, Hydroxyproline, Isoleucine, Leucine, Lysine, Methionine, Phenylalanine, Proline, Serine, Threonine, Tryptophan, Tyrosine, Valine.

Thus, a non-proteogenic amino acid is a moiety which can be incorporated into a peptide via peptide bonds but is not a proteogenic amino acid. Examples are γ-carboxyglutamate, ornithine, phosphoserine, the D-amino acids such as D-alanine and D-glutamine, Synthetic non-proteogenic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), 3-aminomethyl benzoic acid, anthranilic acid, des-amino-Histidine, the beta analogs of amino acids such as β-alanine etc. D-histidine, desamino-histidine, 2-amino-histidine, β-hydroxy-histidine, homohistidine, Na-acetyl-histidine, α-fluoromethyl-histidine, α-methyl-histidine, 3-pyridylalanine, 2-pyridylalanine or 4-pyridylalanine, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid.

The term “analog” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. All amino acids for which the optical isomer is not stated is to be understood to mean the L-isomer. In aspects of the invention a maximum of 17 amino acids have been modified.

The term “derivative” as used herein in relation to a peptide means a chemically modified peptide or an analogue thereof, wherein at least one substituent is not present in the unmodified peptide or an analogue thereof, i.e. a peptide which has been covalently modified.

The term “long-acting insulin” as used herein means a NPH insulin, a degludec insulin, galergine insulin.

In one aspect, the long-acting insulin is NPH insulin. NPH insulin is disclosed in BE435158A and U.S. Pat. No. 2,961,377, which is herein incorporated by reference. NPH Insulin (Neutral Protamine Hagedorn Insulin) is a modified form of regular human insulin. It comprises insulin molecules combined with protamine, a protein that slows down the absorption of insulin when injected under the skin. This modification creates a longer duration of action compared to regular insulin. NPH insulin is known for its intermediate-acting profile, typically lasting for about 12-24 hours. It's useful for maintaining basal insulin levels throughout the day and night.

In one aspect, the long-acting insulin is Degludec insulin. Degludec insulin is disclosed in HUE030383T2 and CA2776296C, which are herein incorporated by reference. Degludec Insulin (Insulin Degludec, Tresiba) is an example of a newer generation of long-acting insulins. Its structure has been modified to form multi-hexamers when injected under the skin. These hexamers provide an ultra-long duration of action, lasting more than 24 hours. The slow and continuous release of insulin mimics the basal insulin secretion in the body, offering stable blood sugar control with a low risk of hypoglycemia. Degludec insulin is known for its flexibility in dosing time, allowing patients to adjust the time of administration without compromising effectiveness.

In one aspect, the long-acting insulin is Glargine insulin. Glargine insulin is disclosed in CA1236012A, which is herein incorporated by reference. Glargine Insulin (Insulin Glargine, Lantus/Toujeo), another long-acting insulin, has a structure that differs from regular insulin. It forms microprecipitates when injected, resulting in a gradual and sustained release of insulin. Glargine typically provides a duration of action of approximately 24 hours, making it suitable for once-daily administration. This insulin analog helps maintain stable blood sugar levels between meals and overnight, reducing the need for multiple daily injections.

The term “excipient” as used herein means the chemical compounds which are normally added to pharmaceutical compositions, e.g. buffers, tonicity agents, preservatives and the like.

The term “effective amount” as used herein means a dosage which is sufficient to be effective for the treatment of the patient compared with no treatment.

The term “pharmaceutical composition” as used herein means a product comprising an active compound or a salt thereof, for example long-acting insulin, together with pharmaceutical excipients such as buffer, preservative, and optionally a tonicity modifier and/or a stabilizer. Thus, a pharmaceutical composition is also known in the art as a pharmaceutical formulation.

### Insulin Compositions

As described above, the present disclosure is directed to a new transdermal delivery system for administration of long acting insulin, such as NPH insulin, in therapeutically effective amounts. This new, external, transdermal administration of NPH insulin provides an alternative way of administering this drug that has not been widely available before. This also provides new new therapeutic possibilities for individuals with obesity and type 2 diabetes.

Transdermal administration of NPH insulin in the form of an external preparation is convenient and can be more acceptable to patients than traditional injection methods or oral intake of medications. This eliminates the need for injections, which can be beneficial for patients with needle phobia and improve overall treatment compliance and convenience. A more convenient administration form can contribute to better adherence to therapeutic recommendations and regular intake of the medication, which is crucial for achieving optimal outcomes in the treatment of obesity and type 1 or 2 diabetes.

The transdermal system with Insulin substance described herein has the capability of penetrating all types of skin. To enhance the transdermal delivery of therapeutic substances, absorption enhancers, also known as permeation enhancers, are introduced into the system, which reversibly alter the structure of the stratum corneum. These are chemical compounds that, when administered together with the therapeutic substance, facilitate its penetration and absorption. The formulation includes a combination of absorption enhancers, such as dimethyl sulfoxide (DMSO), oleic acid, urea or others. This process aims to eliminate the possibility of diffusion limitation and utilize the phenomenon of additive synergy to significantly increase the bioavailability of the therapeutic substance.

The transdermal delivery systems (TDS) described in this context encompass various transdermal formulations, which can be in the form of liquid or semi-solid preparations with a desired viscosity level. These may include solutions, suspensions, nanosuspensions, microsuspensions, dispersions, emulsions, microemulsions, gels, ointments, creams, pastes, lotions, mousses, or balms, among others. Alternatively, the transdermal formulation may constitute a component of a larger TDS.

Examples of such TDS encompass a wide range of applications, including but not limited to topical formulations designed for application to the skin or mucous membranes, whether occlusive or non-occlusive. They may also include gels, lotions, sprays, metered-dose transdermal sprays, aerosols, suppositories, magma formulations, transdermal patches, bilayer matrix patches, multilayer matrix patches, monolithic matrix patches with or without adhesive backing, drug-in-adhesive patches, matrix reservoir patches (featuring a separate matrix reservoir that may optionally be surrounded by adhesive), microreservoir patches, hydrogel matrix patches, mucoadhesive patches, adhesive systems, transdermal tapes, microneedle systems, iontophoresis systems, or combinations thereof.

In some aspects, the percentage content (amount in gr. per 100 gr. of mix) of Insulin in the carrier formulation ranges from 0.03-3%. In the case of the final product dosage form, which is a transdermal patch; the content of Insulin in the patch can range from 0.3 mg to 180 mg.

The dosages for the transdermal and topical systems described here will vary depending on the method of administration, the specific medical condition being treated, and the desired therapeutic effect. The dosing regimen may involve transdermal application once daily for a duration of 1 to 14 days or even longer. Alternatively, it may entail multiple daily applications for a period of 1 to 14 days or more. Another option is to apply the transdermal treatment once every day, every 2 days, every 3 days, every 4 days, every 5 days, every 6 days, every 7 days, every 8 days, every 9 days, every 10 days, every 11 days, every 12 days, every 13 days, or every 14 days.

It is important to note that insulin therapy is a chronic treatment that continues for years. The examples provided here pertain to the duration of a single application of a transdermal patch. Patients will maintain their treatment regimen as necessary by applying subsequent patches for specific periods based on their medical requirements.

Furthermore, the transdermal and topical formulations described here are designed to ensure a consistent absorption rate of NPH insulin (and any other optional medication) by the patient over a predefined period. This predefined period may extend to 24 hours, 48 hours, 72 hours, 96 hours, 120 hours, 144 hours, 7 days, 8 to 13 days, two weeks, or 15 days, depending on specific requirements. Additionally, in certain cases, this absorption rate remains constant.

These formulations can be administered following various dosage regimens, including daily, every two days, every three days, every four days, every five days, every six days, weekly, every 8 to 13 days, bi-weekly, or every 15 to 30 days.

In further aspects, the patient's response to the transdermal delivery systems described herein is assessed through pharmacokinetic analysis of a blood sample. Based on this assessment, adjustments may be made to the transdermal formulations. These adjustments could involve applying smaller or larger patches, multiple transdermal patches, or patches with varying dosages of active ingredients. It's worth noting that the formulations are available in different dosage strengths and patch sizes to ensure the optimal therapeutic outcome tailored to the patient's specific needs.

In various aspects, the formulation may contain one or more additional carriers and excipients, some of which can serve dual or multiple functions. For instance, a specific excipient may act as both a penetration enhancer and a plasticizer, depending on factors like concentration, the type of transdermal system, and its components.

These optional carriers or excipients encompass a wide range of substances, including but not limited to solvents, solubilizers, diluents, agents for suspending and dispersing, gelling agents, polymers, biodegradable polymers, penetration enhancers, plasticizers, pH modifiers, buffering agents, pH stabilizers, emulsifiers, auxiliary emulsifying agents, surfactants, stabilizers, preservatives, chelating agents, complexing agents, emollients, humectants, demulcents, agents to reduce skin irritation, antioxidants, oxidants, tackifiers, fillers, volatile compounds, and materials required for formulating patches or film-like preparations.

In aspects, the formulation may comprise a solvent, e.g. one or more of a C1-C20 alcohol (e.g., without limitation, one or more of: methanol, ethanol, isopropyl alcohol, butanol, propanol, 2-methyl-2-propanol, aka t-butyl alcohol, pentanol, 2,4-dimethyl-2-pentanol, 3,5-dimethyl-3-hexanol, and alcohols having C7, C8, C9, C10, C11, C12, C13, C14, C15, C16, C17, C18, C19 or C20 carbon atoms), polyhydric alcohols, glycols (e.g., without limitation: propylene glycol, polyethylene glycol, dipropylene glycol, hexylene glycol, butyene glycol, glycerine), derivatives of glycols, pyrrolidone (e.g., without limitation: N methyl 2-pyrrolidone, 2-pyrrolidone), sulfoxides (e.g., without limitation: dimethyl sulfoxide aka DMSO and decymethylsulfoxide), dimethylisosorbide, mineral oils, vegetable oils, water, polar solvents, semi polar solvents, non polar solvents, volatile chemicals which can be used to prepare patch formulations such as but not limited to esters, ketones, alcohols, alkanes, such as ethyl acetate, acetone, dichloromethane, chloroform, heptane, hexane, siloxanes, ethanol, isopropanol, toluene, and acids such as acetic acid, lactic acid, levulinic acid, and bases.

In aspects, the formulation may comprise a surfactant, solubilizer, emulsifying agent, or dispersing agent, including anionic, cationic, nonionic and amphoteric surfactants, e.g. one or more of a cetylic alcohol, propylene glycol, monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxyl-6-glycerides, lauroyl polyoxyl-6-gylcerides, polyglyceryl-3-dioleate, diethylene glycol monoethyl ether, propylene glycol monolaurate type I, polyglyceryl-3-dioleate, caprylocaproyl polyoxyl—8 glycerides, cyclodextrins, Diethylene glycol monoethyl ether (DEGEE), a polysorbate/polyethoxylated sorbitan ester or Tween®-type surfactant, a sorbitan ester or Span®-type solvent surfactant, a glycol, hexylengycol, a Brij® type surfactant, and sodium lauryl sulfate. DEGEE (also known as Di(ethylene glycol) ethyl ether or 2-(2-Ethoxyethoxy) ethanol)) is commercially available e.g. under the various trade names including Transcutol® (TC), Transcutol® P, Transcutol® CG, Transcutol® HP (Gattefosse, Lyon, France), and Carbitol™ (Dow Chemicals, Midland Mich.). The Span® or Tween® surfactant may, without limitation, be selected from one or more of: Span 20®, Span®40, Span® 60, Span®80, Span®83, Span®85, Span®120, Tween 20®, Tween 21®, Tween 40®, Tween 60®, Tween 61®, Tween 65®, and Tween 80®. Brij® is a group of nonionic surfactants commercially available from various sources (e.g. Sigma-Aldrich), and may be selected from one or more of Brij® 93 (average Mn ^{~}357), Brij® S 100 (average Mn ^{~}4,670), Brij® 58 (average Mn ^{~}1124), Brij® 010 (average Mn ^{~}709, also known as Brij 97, C18-1E10, Polyoxyethylene (10) oleyl ether, C18H35(OCH2CH2)n0H, n°10), Brij® C10 (average Mn ^{~}683), Brij® L4 (average Mn ^{~}362, also known as polyethylene glycol dodecyl ether, polyoxyethylene (4) lauryl ether, (C20H42O5)n), BRIJ® O20 (average Mn ^{~}1,150, Polyoxyethylene (20) oleyl ether, C18H35(OCH2CH2)nOH, n_{~}20), Brij® S2 MBAL (also known as Brij® S2, polyethylene glycol octadecyl ether, polyoxyethylene (2) stearyl ether, main component: diethylene glycol octadecyl ether, C18H37(OCH2CH2)2OH), Brij® S10 (average Mn ^{~}711), Brij® S20, and Brij® 35 (also known as Brij® L23, C12E23, polyoxyethylene lauryl ether, (C2H4O)nC12H260). Suitable amounts of a surfactant to include into formulations to perform a surfactant function may be from 0.01-95% w/w, less than 5% w/w typically e.g. less than 4, 3, 2, 1, or 0.5%. Suitable amounts for solvent/solubilizing functions may be from 5% to about 50%. Amounts may be increased or decreased to achieve a suitable and sufficient amount, as will be apparent to a person of ordinary skill in the art.

A glycol is class of small organic compounds (e.g. MW typically below 150 Daltons), or a polymer thereof, that belongs to the alcohol family, and wherein two hydroxyl (—OH) groups are attached to different carbon atoms. The simplest member of the glycol class is ethylene glycol (also known as 1,2-ethanediol), other members include, without limitation, propylene glycol (also called 1,2-propanediol), butylene glycol (1,3-butanediol), 1,4-butanediol, pentylene glycol, (1,2-pentanediol), hexylene glycol (2,4-pentanediol), 2-ethyl-1,3-hexanediol, and 2-methyl-2-propyl-1,3-propanediol. Similarly, higher molecular weight polymers of the above glycol diols, in particular of ethylene glycol, may be used; these include, without limitation, polyethyleneglycol (PEG). PEGs are available in different molecular weights, typically from about 200 g/mol to about 10,000,000 g/mol, e.g. PEG 200, 300, 400, 600, 800, 1000, 1500, 3350, 4000, 6000, 8000, 10,000, 20,000, 35,000. PEGs of different molecular weight have similar surfactant properties but the higher molecular weight polymers may be preferred for their additional thickening function which may be desired in some patch formulations.

A formulation for use in the methods of this invention may incorporate a permeation enhancer. Numerous penetration enhancers comprising a variety of structurally diverse compounds are known and can be used individually or in combination, as evident to a person skilled in the art.

Examples of penetration enhancers encompass alcohols such as ethanol, propanol, isopropanol, nathanol, dodecanol, propylene glycol, and glycerol, as well as ether alcohols like diethylene glycol monoethyl ether. Additionally, fatty acids, fatty alcohols, fatty acid derivatives, fatty alcohol derivatives, sulfoxides (e.g., dimethylsulfoxide, decylmethyl sulfoxide), amides (e.g., dimethylformamide, azone, urea, dimethylacetamide), pyrrolidone derivatives (e.g., 1-methyl-4-carboxy-2-pyrrolidone, 1-methyl-2-pyrrolidone, 1-lauryl-4-methoxycarbonyl-2-pyrrolidone), terpenes (e.g., menthol, limonene, terpineol, pinene, carvol), terpenoids, essential oils (e.g., tee tree oil, lavender oil), ethyl acetate, methyl acetate, octisalate, pentadecalactone, and acrylamide are considered as potential penetration enhancers.

Moreover, triglycerides (e.g., triacetin), polyoxyethylene fatty alcohol ethers, polyoxyethylene fatty acid esters, esters of fatty alcohols, essential oils, and surfactant-type enhancers like brij, sodium lauryl sulfate, tween, and polysorbate may be utilized as well. Any other penetration or permeation enhancers listed in the book “Percutaneous Penetration Enhancers” (Eric W. Smith, Howard I. Maibach, 2005. November, CRC press) may also be included.

In certain aspects, the permeation enhancer and mixture of thereof may be present in the range of 0.01% to 97% w/w.

Fatty alcohol permeation enhancers comprise various saturated, monounsaturated, or polyunsaturated fatty alcohols, including but not limited to the following: Butanol (C4), Butyl alcohol (C4), Tert-butyl alcohol (C4), Tert-amyl alcohol (C5), 3-Methyl-3-pentanol (C6), Capryl alcohol (C8), Pelargonic alcohol (C9), Capric alcohol (C10), Undecyl alcohol (C11), Lauryl alcohol (C12), Tridecyl alcohol (C13), Myristyl alcohol (C14), Pentadecyl alcohol (C15), Cetyl alcohol (C16), Palmitoleyl alcohol (cis-9-hexadecen-1-ol, C16H320), Heptadecyl alcohol (1-n-heptadecanol, C17H36O), Stearyl alcohol (C18:0), Oleyl alcohol (C18H36O, C18:1), Linoleyl alcohol (C18H34O, cis,cis-9,12-Octadecadien-1-ol), Nonadecyl alcohol (C19), Arachidyl alcohol (C20H42O), Octyldodecanol (C20H42O, 2-Octyldodecan-1-ol), Heneicosyl alcohol (C21), Behenyl alcohol (C22H46O), Erucyl alcohol (cis-13-docosen-1-ol, C22H440), Lignoceryl alcohol (C24), Ceryl alcohol (C26)

Saturated fatty alcohol permeation enhancers include, but are not limited to: Lauryl alcohol (C12), Isolauryl alcohol (C12, 10-methyl-1-hendecanol), Anteisolauryl alcohol (C12, 9-methyl-1-hendecanol), Myristyl alcohol (C14), Isomyristyl alcohol (C14, 12-methyl-1-tridecanol), Anteisomyristyl alcohol (C14, 11-methyl-1-tridecanol), Cetyl alcohol (C16), Isopalmityl alcohol (C16, 14-methyl-1-pentadecanol), Anteisopalmityl alcohol (C16, 13-methyl-1-pentadecanol), Stearyl alcohol (C18), Isostearyl alcohol (C18, 16-methyl-1-heptadecanol), Anteisostearyl alcohol (C18, 15-methyl-1-pentadecanol).

Fatty alcohol or acid permeation enhancers for the described formulations include, but are not limited to: Oleic acid, Ethyl oleate (OA ethyl ester), Oleyl oleate (OA oleyl ester, C36H68O2), Glyceryl oleate (OA glyceryl ester), Decyl oleate, Sorbitan monooleate (sorbitan oleate, Span 80), Glycerol monooleate, Oleyl alcohol (cis-9-octadecen-1-ol), Elaidic acid (C18:1), Gondoic acid (C20:1), Erucic acid (C22:1), Nervonic acid (C24:1), Ximenic acid (C26:1), Chaulmoogra acid, Hydnocarpic acid or derivatives thereof. Additionally, polyunsaturated acids, such as: Hexadecatrienoic acid (16:3), Linoleic acid (C18:2), Alpha-linolenic acid (C18:3), Gamma-linolenic acid (C18:3), Calendic acid (C18:3), Stearidonic acid (C18:4), Mead acid (C20:3), Eicosadienoic acid (C20:3), Eicosatrienoic acid (C20:3), Dihomo-gamma-linolenic acid (C20:3), Arachidonic acid (C20:4), Docosadienoic acid (C22:2), or derivatives thereof, including alcohols and esters, e.g., linoleyl alcohol (the fatty alcohol of linoleic acid). In some aspects, these fatty alcohols and acids are among the permeation enhancers used in the formulations described above.

Additional optional excipients may include, among others, one or more pH adjusting and buffering agents selected from the following options:
Buffers (e.g., citrate buffer, phosphate buffer, acetate buffer)
Acids and acid derivatives (e.g., carboxylic acid, organic acid, inorganic acid, sulfonic acid, halogenated carboxylic acids, vinylogous carboxylic acids, hydrochloric acid, acetic acid, succinic acid, citric acid, ascorbic acid, phosphoric acid)
Bases and base derivatives (e.g., sodium bicarbonate, sodium carbonate, dimethylamine, trimethylamine, triethanolamine, sodium hydroxide, calcium hydroxide, potassium hydroxide, ammonium hydroxide, tromethamine).

In some aspects, weak organic acids or weak organic bases are used as pH adjusting agents. The pH adjusting/buffering agent or stabilizer plays a crucial role in maintaining the appropriate pH of the transdermal formulation.

Moreover, additional optional excipients encompass, without restriction, one or more of the following: emulsifying agents, auxiliary emulsifying agents, surfactants, suspending agents, preservatives, antioxidants, chelating agents, emollients, humectants, demulcents, agents to reduce skin irritation, tackifiers, fillers, cross-linking agents, resins, crystallization inhibitors, and clays.

Matrix-forming or gel-forming polymers may be used to form a transdermal gel, reservoir patch or matrix patch, and a large number of such polymers may be employed alone or in combination in amounts depending on the particular delivery vehicle and intended use (e.g. viscosity, duration of application, adherence etc.) as will be apparent to a person of ordinary skill. Exemplary polymers include, without limitation, cellulose and its derivatives (such as but not limited to hydroxy methyl cellulose, Aquasolve™ hypermellose acetate succinate, hydroxypropyl methyl cellulose, hydoxypropyl cellulose, ethyl cellulose, hydroxyethyl cellulose, methyl cellulose, carboxymethyl cellulose, sodium carboxymethyl cellulose, microcrystalline cellulose blends, cellulose acetate phthalate, propylmethylcellulose phthalate, etc.), biodegradable polymers (such as but not limited to gelatin, chitosan, starch, polyacrylic acid, polyvinyl, etc.), gums (such as but not limited to guar gum, gum copal, gellum gum, xanthan gum, locust bean gum, gum arabic, tragacanth, cassia gum, karaya gum etc.), polysaccharides (such as but not limited to carrageenan, agar, pectin, mannan, alginic acid, dextran, pullulan, etc.).

Film forming, rheological property modifying and or thickening polymers such as but not limited to starch and its derivatives, gelatin and its derivatives, polyvinyl alcohol and its derivatives, polyvinylpyrrolidone and its derivatives, ethylene and its derivatives, propylene and its derivatives, ethylene-vinyl acetate (EVA) and its derivatives. Film forming polymeric materials include methylcellulose and its derivatives, microcrystalline cellulose and its derivatives, cellulose and its derivatives, acrylates and copolymers thereof. Specific examples of these materials includes Kollidon® 12, 17, 30, 90 and VA64 from BASF. Additional examples include Elvanol from Kuraray. Pure-Cote®, Instant Pure-Cote®, Pure-Gel® and Pure-Dent® are various starch grades from Grain Processing Corporation. Specific examples of EVA include Ateva® Standard grades obtained from Celanese, ammonioalkyl methacrylate copolymers (e.g. EUDRAGIT® L100-55, EUDRAGIT® E PO, EUDRAGIT® RL, EUDRAGIT ER 100, PLASTOID® B, dimethylaminoethyl methacrylate-butyl methacrylate-methyl methacrylate copolymer).

Controlling the transport process of Insulin group substances (NPH Insulin) is based on modeling the rate and depth of penetration, as well as the possibility of absorption into the bloodstream.

The control of active substance transport is multi-level and based on several elements, namely the use of a combination of different excipients, particularly absorption enhancers, in various concentrations and proportions allows for control over the rate, depth, and duration of absorption; and the selection of proportions and concentrations of excipients forming the carrier depends on the active substance (Insulin) and its physicochemical character and target action profile.

The proportions of absorption enhancers and the appropriately selected pH of the formulation influence the degree and duration of intercellular softening (temporary destabilization of the three-layered intercellular structure), particularly between keratinocytes and corneocytes of the epidermis.

The appropriately selected pH of the formulation affects the degree of cell membrane polarization, particularly the transport of substances through ion channels into the interior of the cells.

In systemic application, the anti-diabetic preparation containing Insulin, preferably NPH Insulin, with the appropriate proportion of absorption enhancers, pH, and quantity of sodium and potassium ions, will cause the opening of ion channels and accelerated absorption into the bloodstream.

Method of incorporation of the therapeutic substance: The therapeutic substance can be introduced into the carrier in several ways: as a solution (always non-aqueous), in suspension, with the formation of micro or nano micelles, as a supersaturated solution. Each of these methods results in different parameters of absorption and release of the therapeutic substance into the organism. For example, introducing a supersaturated solution results in a multi-phase system, where part of the active substance is enclosed in a non-aqueous micellar structure and constitutes an initiating dose, rapidly absorbed into the organism and saturating individual barrier layers. Universality is considered in several aspects.

Preparations based on this principle can be applied in the form of ointments or creams. A semi-solid carrier can be part of a transdermal system—a patch. After thickening, it can also serve as a base for suppository formation.

The acceptable pharmaceutical carrier includes excipients with the character of polar solvents (for substances soluble in water) and non-polar solvents (for substances soluble in fats). These solvents form the structure of the carrier through the use of emulsifiers with high biocompatibility. Increased stability of the active substance is achieved by maximizing the reduction of the aqueous fraction during the carrier manufacturing process and during the incorporation process.

The pharmaceutical composition characterized by containing a pharmaceutical carrier as defined below and a therapeutic substance from the Insulin group (e.g., NPH Insulin), wherein the ratio of the therapeutic substance from the Insulin group (e.g., NPH Insulin) to the pharmaceutical carrier is from 0.03-95% to 95-0.03%. Additionally, advantageously, the composition according to the invention is characterized by containing a excipients and it takes the form of a semi-solid dosage form.

### Disease Conditions

The present disclosure provides methods for the prevention and treatment of type 2 diabetes, obesity, or a related condition. The term, “treatment” as used herein, refers not only to ameliorating symptoms associated with the disease, but also preventing or delaying the onset of the disease, and also lessening the severity or frequency of symptoms of the disease, preventing or delaying the occurrence of a second episode of the disease or condition; and/or also lessening the severity or frequency of symptoms of the disease or condition. The main therapeutic effects is lowering blood glucose level phenomenon.

Along with Insulin resistance, hypertension, and dyslipidemia, obesity is considered to be a component of the Metabolic Syndrome (also known as Syndrome X) which together synergize to potentiate cardiovascular disease. More recently, the U.S. National Cholesterol Education Program has classified Metabolic Syndrome as meeting three out of the following five criteria: fasting glucose level of at least 110 mg/dl, plasma triglyceride level of at least 150 mg/dl (hypertriglycerdemia), HDL cholesterol below 40 mg/dl in men or below 50 mg/dl in women, blood pressure at least 130/85 mm Hg (hypertension), and central obesity, with central obesity being defined as abdominal waist circumference greater than 40 inches for men and greater than 35 inches for women. The American Diabetes Association estimates that 1 in every 5 overweight people suffer from Metabolic Syndrome.

According to the guidelines of the American Diabetes Association, to be diagnosed with Type 2 diabetes, an individual must have a fasting plasma glucose level greater than or equal to 126 mg/dl or a 2-hour oral glucose tolerance test (OGTT) plasma glucose value of greater than or equal to 200 mg/dl (Diabetes Care, 26: S5-S20, 2003). A related condition called pre-diabetes is defined as having a fasting glucose level of greater than 100 mg/dl but less than 126 mg/dl or a 2-hour OGTT plasma glucose level of greater than 140 mg/dl but less than 200 mg/dl. Mounting evidence suggests that the pre-diabetes condition may be a risk factor for developing cardiovascular disease (Diabetes Care 26:2910-2914, 2003). Prediabetes, also referred to as impaired glucose tolerance or impaired fasting glucose is a major risk factor for the development of type 2 diabetes mellitus, cardiovascular disease and mortality.

### EXAMPLES

In the performance examples, the mixtures used in the tests are described and it is preferred that the components used are selected from the groups as described below.

Excipient 1 selected from the group consisting of fatty alcohol, terpenes, essential oils or acid permeation enhancers (e.g. Lauryl alcohol (C12), Isolauryl alcohol (C12, 10-methyl-1-hendecanol), Anteisolauryl alcohol (C12, 9-methyl-1-hendecanol), Myristyl alcohol (C14), Isomyristyl alcohol (C14, 12-methyl-1-tridecanol), Anteisomyristyl alcohol (C14, 11-methyl-1-tridecanol), Cetyl alcohol (C16), Isopalmityl alcohol (C16, 14-methyl-1-pentadecanol), Anteisopalmityl alcohol (C16, 13-methyl-1-pentadecanol), Stearyl alcohol (C18), Isostearyl alcohol (C18, 16-methyl-1-heptadecanol), Anteisostearyl alcohol (C18, 15-methyl-1-pentadecanol), Oleic acid, Ethyl oleate (OA ethyl ester), Oleyl oleate (OA oleyl ester, C36H68O2), Glyceryl oleate (OA glyceryl ester), Decyl oleate, Sorbitan monooleate (sorbitan oleate, Span 80), Glycerol monooleate, Cholesterol, Oleyl alcohol (cis-9-octadecen-1-ol), Chaulmoogra acid, Hydnocarpic acid, terpenes (e.g., menthol, limonene, terpineol, pinene, carvol), terpenoids, Lavender oil, tee tree oil, Adeps Suillus, Adeps Solidus, Chaulmoogra oil, sunflower oil, flaxseed oil, rapeseed oil, peanut oil, olive oil or is a mixture of each of these.

Excipient 2 selected from the group consisting of sulfoxides (e.g., dimethylsulfoxide, decylmethyl sulfoxide), amides (e.g., dimethylformamide, azone, urea, dimethylacetamide), pyrrolidone derivatives (e.g., 1-methyl-4-carboxy-2-pyrrolidone, 1-methyl-2-pyrrolidone, 1-lauryl-4-methoxycarbonyl-2-pyrrolidone), ethyl acetate, methyl acetate, octisalate, pentadecalactone, Choline chloride, L-Proline, and acrylamide or is a mixture of each of these.;

Excipient 3 selected from the group consisting of surfactants: Span 20®, Span®40, Span® 60, Span®80, Span®83, Span®85, Span®120, Tween 20®, Tween 21®, Tween 40®, Tween 60®, Tween 61®, Tween 65®, and Tween 80® or is a mixture of each of these.

A solvent was selected from the group consisting of one or more of a C1-C20 alcohol (e.g., without limitation, one or more of: methanol, ethanol, isopropyl alcohol, butanol, propanol, 2-methyl-2-propanol, aka t-butyl alcohol, pentanol, 2,4-dimethyl-2-pentanol, 3,5-dimethyl-3-hexanol, and alcohols having C7, C8, C9, C10, C11, C12, C13, C14, C15, C16, C17, C18, C19 or C20 carbon atoms), polyhydric alcohols, glycols (e.g., without limitation: propylene glycol, polyethylene glycol, dipropylene glycol, hexylene glycol, butyene glycol, glycerine), derivatives of glycols, pyrrolidone (e.g., without limitation: N methyl 2-pyrrolidone, 2-pyrrolidone) or is a mixture of each of these.

### Example 1

A NPH Insulin formulation is prepared by mixing a pharmaceutically acceptable form of NPH Insulin, with one or more excipients (or enhancers), and a solvent. The proportions of each by % w/w are shown below.

| Formulation | % w/w |
|---|---|
| NPH Insulin | 0.03-3% |
| Excipient 1 | 45-65% |
| Excipient 2 | 8-25% |
| Excipient 3 | 0.1-10% |
| Solvent | 10-35% |
| Total | 100 |

### Example 2

A degludec insulin formulation is prepared by mixing a pharmaceutically acceptable form of degludec insulin, with one or more excipients (or enhancers), and a solvent. The proportions of each by % w/w are shown below.

| Formulation | % w/w |
|---|---|
| degludec insulin | 0.03-3% |
| Excipient 1 | 45-65% |
| Excipient 2 | 8-25% |
| Excipient 3 | 0.1-10% |
| Solvent | 10-35% |
| Total | 100 |

### Example 3

A glargine insulin formulation is prepared by mixing a pharmaceutically acceptable form of glargine insulin, with one or more excipients (or enhancers), and a solvent. The proportions of each by % w/w are shown below.

| Formulation | % w/w |
|---|---|
| glargine insulin | 0.03-3% |
| Excipient 1 | 45-65% |
| Excipient 2 | 8-25% |
| Excipient 3 | 0.1-10% |
| Solvent | 10-35% |
| Total | 100 |

### Example 4

A detemir insulin formulation is prepared by mixing a pharmaceutically acceptable form of detemir insulin, with one or more excipients (or enhancers), and a solvent. The proportions of each by % w/w are shown below.

| Formulation | % w/w |
|---|---|
| detemir insulin | 0.03-3% |
| Excipient 1 | 45-65% |
| Excipient 2 | 8-25% |
| Excipient 3 | 0.1-10% |
| Solvent | 10-35% |
| Total | 100 |

The pharmaceutical carrier for the therapeutic substance from the Insulin group (e.g., NPH Insulin) is characterized by containing a strongly solvating aprotic solvent from the sulfoxide group, a carbamic acid amide, and a vegetable oil containing unsaturated fatty acids. In one aspect, the pharmaceutical composition of the present disclosure is characterized by a strongly solvating aprotic solvent from the sulfoxide group selected from the group containing dimethyl sulfoxide. Likewise, advantageously, the carrier according to the invention is characterized by the carbamic acid amide selected from the group of absorption enhancers containing urea. In another advantageous aspect of the invention, the pharmaceutical composition is characterized by the vegetable oil with a high content of unsaturated fatty acids selected from the group containing Adeps Suilus, Adeps Solidus, Chaulmoogra oil, sunflower oil, plant oils, preferably flaxseed oil, rapeseed oil, peanut oil, olive oil.

In another aspect of the disclosure, the carrier is characterized by containing the solvating aprotic solvent from the sulfoxide group. Moreover, the carrier according to the invention is advantageously characterized by containing the carbamic acid amide. Similarly, the carrier according to the invention is advantageously characterized by containing the vegetable or mineral oil. The most advantageously, the carrier according to the invention is characterized by containing an emulsifying substance in an amount of 0.1% to 10% by weight of the drug.

The composition is characterized by the fact that the excipient of the pharmaceutical carrier are selected from the group consisting of Adeps Suilus (lard), and/or Adeps Solidus, and/or Eucerin, preferably white petrolatum, yellow petrolatum, and/or zinc sulfate-zinc salts, their arbitrary combinations, and most preferably lard, Eucerin, and zinc sulfate.

The mixture of excipients forms a carrier characterized by its impact on the bioavailability of active substances. For example, a semi-solid carrier for active substances was prepared using the following excipients: A, preferably Dimethylsulfoxide (DMSO), B, preferably Urea, C, preferably Oleic Acid and D, cetylic alcohol.

The mixture of substances constituting the carrier consists of elements ensuring the dissolution or emulsification of both water-soluble active substances and lipid-soluble active substances. The dissolution of hydrophilic active substances is facilitated by the A-B mixture. The dissolution of lipophilic substances is facilitated by the excipient C. The A-B-C mixture, preferably with the addition of D, is responsible for the emulsifying properties.

### Example 5

The mixture serving as a carrier for substances from the Insulin group (e.g., NPH Insulin) was prepared in the following stages:
a) Adeps Suilus (lard) with cetylic alcohol, cholesterol and oleic acid was melted in a vessel at a maximum temperature of 40 °C to create a lipophilic phase (mixture A).
b) To the mixture B (hydrophilic phase) of propylene glycol, Choline Chloride and Urea strongly solvating aprotic solvent from the group of sulfoxides was added while continuously stirring.
c) To the mixture B and strongly solvating aprotic solvent from the group of sulfoxides, an appropriate amount of surfactant was added.
d) The mixture A and mixture B was thoroughly mixed until a mass with a homogeneous consistency and color was obtained.
e) The resulting final mixture (A+B+surfactant) exhibited emulsifying properties. In the melted mixture, substances with a lipophilic character dissolved. In the final mixture at room temperature, substances with a hydrophilic character dissolved.
f) The subject of the disclosure is the utilization of the described carrier in a semi-solid form of a drug along with a unique mixture of active substances in a carefully selected proportion.

The active substances encompass all synthetic and naturally occurring substances from the Insulin group (e.g., NPH Insulin).

The weight proportions between the active substances of the Insulin group and the carrier should fall within the range of 0.03-95% to 95-0.03%. The percentage provided represents the concentration in 100 parts of the final product. The carrier constitutes from 0.1% to 95% by weight of the final product. The pharmaceutical composition of the carrier can consist of lipophilic base obtained from Adeps Suillus or Adeps Solidus ranging from 45% to 65%. The weight ratio of active substances from the Insulin group to excipients ranges from 0.03-95%, and it takes the form of a semi-solid drug form characterized by including:
a) Mixing hydrophilic substances with a portion of active substances from the Insulin group to create levigations, in some aspects from 0.03% to 5%, of the necessary carrier for the entire process, within a temperature range of 20-50 °C, in pharmaceutical and food mixers at up to 4000 RPM, for a minimum of 3 minutes.
b) Mixing the resulting mixture from step a) with a portion, in some aspects from 6% to 90%, of the remaining amount of carrier needed for the entire process, within a temperature range of 20-50 °C, in pharmaceutical and food mixers at up to 5000 RPM, for a minimum of 3 minutes.
c) Mixing the resulting mixture from step b) with the remaining amount of carrier needed for the entire process and then with the remaining auxiliary substances to achieve homogeneity, within a temperature range of 20-50 °C, in pharmaceutical and food mixers at up to 4000 RPM, for a minimum of 3 minutes.
d) Homogenizing in laboratory and industrial homogenizers (rotary and piston) within a temperature range of 20-50 °C, for a minimum of 5 minutes.

Thus, a homogeneous mixture of the carrier with active pharmaceutical substances is created, containing evenly dispersed active substances in a semi-solid mass.

### Example 6

The compositions described above can be used in treatment regimens as described below:
A. One transdermal therapeutic system patch applied daily containing 0.3 mg to 180 mg of the therapeutic substance. (Example 1)
B. One transdermal therapeutic system patch applied for 8 hours containing from 0.3 mg to 10.8 mg of the therapeutic substance. Another transdermal therapeutic system patch applied after another 16 hours.
C. One transdermal therapeutic system patch applied for 3 to 6 days containing from 10.8 mg to 180 mg of the therapeutic substance. Another transdermal therapeutic system patch applied in day 7.
D. One transdermal therapeutic system patch applied every 7 to 14 days containing 10.8 mg to 180 mg of the therapeutic substance. The next transdermal therapeutic system patch applied immediately after the previous application.

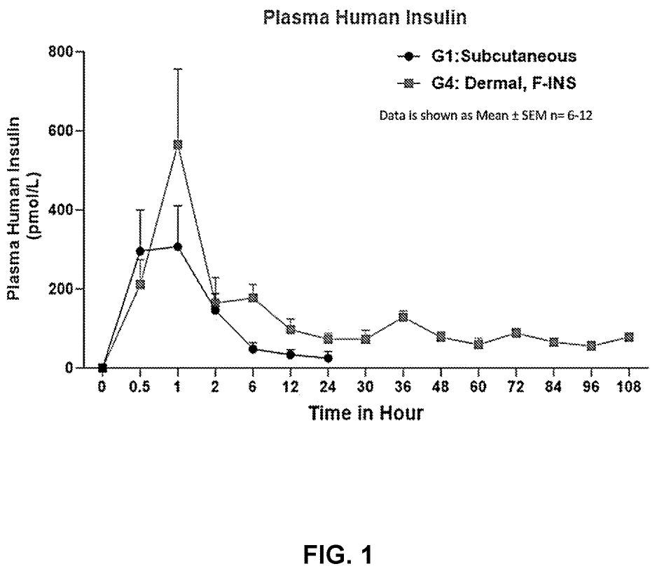

The described invention was validated during an in vivo experiment on laboratory animals (rats). A formulation containing NPH insulin was made according to the composition described in Example 1 and the manufacturing procedure described in Example 5. One transdermal therapeutic system patch applied for 3 days containing 0.36 mg of the therapeutic substance. The transdermal form (described in the experiment under the designation—G4. Dermal. F-INS) was compared to the classic subcutaneous injection of NPH insulin (described in the experiment under the designation—G1. Subcutaneous). The results of the experiment are shown in FIG. 1, a comparison of the pharmacokinetic parameters of the two forms of NPH insulin administration is presented in Table 1.

TABLE 1
Comparison of pharmacokinetic parameters of human insulin after
transdermal administration and classical subcutaneous injection.

| | Group 1, | Group 3, |
|---|---|---|
| Parameter | Subcutaneous | Dermal - F-INS |
| Dose D [mg/Rat]= | 0.035 | 0.350 |
| T_{1/2} [h]= | 19.7 | |
| C_{MAX} [pmol/L]= | 306.94 | 565.81 |
| T_{MAX} [h]= | 1.0 | 1.0 |
| MRT [h]= | 21.8 | 62.3 |
| AUC_{0→t=24} [pmol · h/L]= | 1439.9 | 3142.4 |
| AUC_{INF} [pmol · h/L]= | 2146.4 | 11820.5 |

| | |
|---|---|
| AUC_{INF}_{_{—}}_{TTS}/AUC_{INF}_{_{—}}_{SC}= | 5.51 |
| AUC_{0→t=24}_{_{—}}_{TTS}/AUC_{0→t=24}_{_{—}}_{SC}= | 2.18 |
| F_{REL}_{_{—}}_{INF} [%]= | 55.1 |
| F_{REL}_{_{—}}_{0→t=24} [%]= | 21.8 |

The results presented here demonstrate, in a clear and unambiguous manner, the advantages of using the transdermal form of insulin over the currently used subcutaneous injection form. Following the administration of a transdermal system containing insulin NPH over a 72-hour period, a constant insulin level is maintained until 108 hours after application, which falls within the therapeutic window. It is crucial to highlight that the results of the experiment unequivocally demonstrate that there is no delay in the therapeutic effect when comparing the concentration profiles after transdermal administration to the classic subcutaneous administration of NPH insulin. The potential to administer insulin transdermally using described solution offers a significant improvement in patient comfort and compliance, reducing the need for daily injections.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

What is claimed is:

1. A pharmaceutical composition comprising an Insulin and a pharmaceutical carrier selected from dimethyl sulfoxide (DMSO), urea, propylene glycol, L-Proline, Adeps Suilus, oleic acid, Choline or their salts, Cholesterol, Tween 80, Span 80, cetylic alcohol, essential oils, vegetable oil, mineral oil or combinations thereof.

2. The pharmaceutical composition of claim 1, wherein the Insulin is selected from the group consisting of NPH insulin, glargine insulin, degludec insulin, detemir insulin.

3. The pharmaceutical composition of claim 1 or 2, wherein the insulin is NPH insulin.

4. The pharmaceutical composition of any one of claims 1-3 which is formulated for transdermal delivery.

5. The pharmaceutical composition of any one of claims 1-4, wherein composition comprises a transdermal liquid formulation, a transdermal semisolid formulation, a transdermal polymer matrix formulation, or combinations thereof.

6. The pharmaceutical composition of any one of claims 1-5, wherein the composition is in the form of a transdermal patch.

7. The pharmaceutical composition of claim 6, wherein the transdermal patch is selected from the group consisting of a reservoir patch, a micro reservoir patch, a matrix patch, a pressure sensitive adhesive patch, and an extended-release transdermal film.

8. A method of transporting an Insulin through the skin and into systemic circulation comprising: administering to a subject in need of the treatment a formulation comprising the pharmaceutical composition of any one of claims 1-7.

9. The method of claim 8, wherein administration of the formulation comprising an Insulin and the pharmaceutically acceptable carrier comprises continuous administration of the formulation to the subject for one day, two days, three days, four days, five days, six days, seven days, eight days, nine days, ten days, eleven days, twelve days, thirteen days, or fourteen days.

10. A method of treating diabetes in a subject, comprising transdermally administering a therapeutically effective amount of the pharmaceutical composition of any one of claims 1-7 to a subject in need thereof.

11. A method of treating obesity in a subject, comprising transdermally administering a therapeutically effective amount of the pharmaceutical composition of any one of claims 1-7 to the subject in need thereof.

12. Use of a pharmaceutical composition comprising an Insulin and a pharmaceutical carrier selected from dimethyl sulfoxide (DMSO), urea, propylene glycol, L-Proline, Adeps Suilus, oleic acid, Choline or their salts, Cholesterol, Tween 80, Span 80, cetylic alcohol, essential oils, vegetable oil, mineral oil or combinations thereof, in the treatment of diabetes.
